(19) United States
(12) Patent Application Publication
(10) Pub. No.: US 2009/0069615 A1
(43) Pub. Date: Mar. 12, 2009

(54) METHOD AND EQUIPMENT FOR PREPARING ISOBUTYLENE USING TERTIARY BUTYL ALCOHOL

(75) Inventors: Jianqiu Yu, Hong Kong (CN); Deyu Chen, Fuzhou City (CN); Junxiong Li, Fuzhou City (CN); Zihong Chen, Fuzhou City (CN); Gonghao Chen, Fuzhou City (CN); Qinyu Huang, Fuzhou City (CN); Caiyuan Chen, Fuzhou City (CN)

Correspondence Address: SUGHRUE MION, PLLC, 2100 PENNSYLVANIA AVENUE, N.W., SUITE 800, WASHINGTON, DC, 20037, US

(73) Assignee: Sichuan Gushan Oil Chemical Co. Ltd., Hong Kong (CN)

(21) Appl. No.: 12/204,588

(22) Filed: Sep. 4, 2008

(30) Foreign Application Priority Data
Sep. 6, 2007 (CN) 200710049954.1

(51) Int. Cl. C07C 1/20; B01J 19/00

(52) U.S. Cl. 585/639; 422/187

(57) ABSTRACT

This invention discloses a method and equipment for preparing isobutylene using tertiary butyl alcohol. The equipment comprises a reaction vessel and a separating column. The upper portion of the separating column is fitted with a column top condenser, and the bottom of the column is fitted with a heater. The reaction vessel and the separating column are connected to each other by means of a pipe. One end of the dehydration column is connected to the heater at the bottom of the separating column, and the other end, after passing through a cooling unit, is connected to a distributor at the top of the separating column. A condenser with an outlet is connected to the outside of the said column top condenser. Isobutylene gas, which is generated as the solid superacid catalyst continuously reacts with and splits the tertiary butyl alcohol being introduced into the reaction vessel, flows together with boiling tertiary butyl alcohol gas into the separating column and is conducted by the distributor to the dehydrated extractant. Isobutylene gas flows out of the top of the column and passes through the condenser whereby it is cooled to become liquid isobutylene finished product. Unreacted tertiary butyl alcohol is condensed by the column top condenser to form liquid condensate, part of which is refluxed within the column and part of which flows out, returning to the reaction vessel. The dehydrated extractant passes through the distributor and enters the separating column from the top of the column. After the moisture in the rising gas is extracted and separated, it flows to the bottom of the column. After the dehydrated extractant has absorbed water, it is sent to the dehydration column where it is heated, the steam is separated from it, and the product is obtained.

### TECHNICAL FIELD

This invention pertains to a method and equipment for preparing isobutylene using tertiary butyl alcohol.

### BACKGROUND ART

Isobutylene is a raw material of organic synthesis that has a broad range of uses. Because it has a low boiling point, it has to be stored and transported in the form of liquefied gas in steel bottles or pressurized tanks and is generally used in specialized factory settings. There are a large number of patented technologies whereby the isobutylene in the C4 fraction at large-scale petrochemical factories is converted to tertiary butyl alcohol by means of hydration and then the tertiary butyl alcohol is cracked to make isobutylene. But in all of these cases, high-pressure equipment is required, there is a great deal of danger involved, and the investment is large; and none are appropriate for self-production of isobutylene for in-house use by mid- to small-scale users.

### CONTENT OF INVENTION

1. Purpose of the Invention:

This invention is geared towards remedying the insufficiency of existing technological solutions by providing specialized equipment and a method of using tertiary butyl alcohol to prepare isobutylene that can be employed at atmospheric pressure, requires low investment, and involves a low degree of danger.

2. Technical Solution:

A method of preparing isobutylene using tertiary butyl alcohol, which includes the following steps sequentially: continuously adding the raw material tertiary butyl alcohol to a reaction vessel containing a solid superacid catalyst sold on the market and heating until a rolling boil and cracking begins; the isobutylene gas produced by cracking and the boiling tertiary butyl alcohol gas flow together into a separating column in which the top contains a condenser and the bottom contains a heater; conducting the dehydrated extractant from the distributor into the separating column and performing separation, thereby causing the isobutylene gas to flow out of the top of the separating column and to be cooled by the condenser to yield liquid isobutylene finished product; under the action of the condenser at the top of the separating column, unreacted tertiary butyl alcohol is condensed to become a condensed liquid, part of which is refluxed within the column and part of which flows out and returns to the reaction vessel; from the distributor, the dehydrated extractant enters the separating column from the top and, after the water is separated by the extractant from the rising gas, it flows to the bottom of the column; after the dehydrated extractant has absorbed water, it is sent to a dehydration column where it is heated and steam is extracted from it, after which a pump is used to send it to the condenser to be cooled, and then it is returned to the separating column, where it continues to participate in the reaction.

Equipment for preparing isobutylene using tertiary butyl alcohol, which comprises a reaction vessel 1 and a separating column 4, characterized in that the upper part of the said separating column 4 is fitted with a column top condenser 2 and the bottom of the column is fitted with a heater 5; the said reaction vessel 1 with a raw material feed inlet and the separating column 4 are connected using a pipe; one end of the dehydration column 6, which has an outlet, is connected to the heater 5 at the bottom of the separating column 4, and the other end, after passing through a cooling unit 8, is connected to a distributor 3 at the top of the separating column; a condenser 7 with an outlet is connected to the outside of the said column top condenser 2.

3. Beneficial Effects:

The technology and equipment used in this invention are all very simple and, furthermore, the equipment functions at atmospheric pressure, the degree of danger is low, and the equipment involves a small investment; these technology and equipment are fully capable of realizing the goal of this invention. They are especially appropriate for self-production of isobutylene for in-house use by mid- to small-scale users.

### DESCRIPTION OF ATTACHED DRAWINGS

The drawings attached to the Description are an embodiment of this invention.

FIG. 1 is a technology flowchart of the production method of this invention.

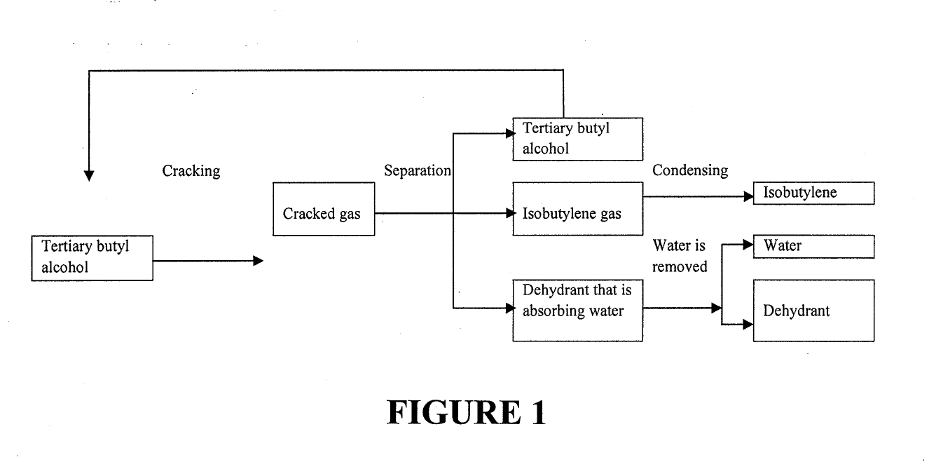

FIG. 2 is a structural diagram of the specialized equipment used in this invention.

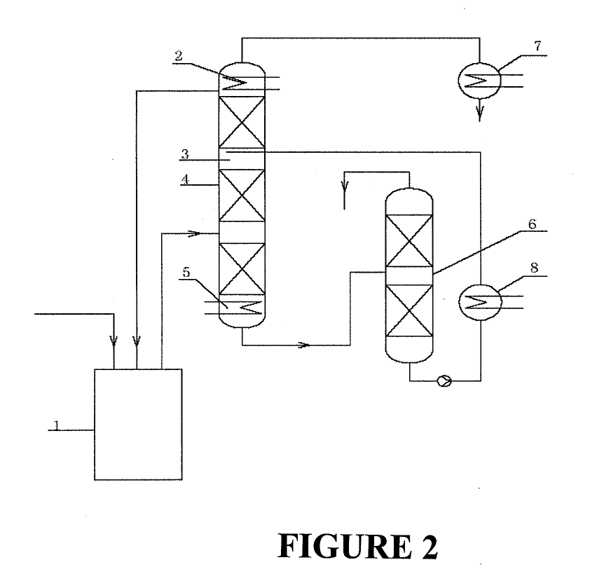

In this figure, 1 is the reaction vessel, 2 is the column top condenser, 3 is the distributor, 4 is the separating column, 5 is the heater at the bottom of the separating column, 6 is the dehydration column, 7 is the condenser, and 8 is the cooling unit.

### Specific Embodiment

A reaction vessel 1 is charged with solid superacid catalyst which is sold on the market; the raw material tertiary butyl alcohol is continuously added to reaction vessel 1 and heated until it is at a rolling boil. Isobutylene gas generated by cracking and boiling tertiary butyl alcohol gas flow together into separating column 4. The top of the separating column is fitted with a column top condenser 2, and the bottom is fitted with a heater 5. The dehydrated extractant is conducted in from the distributor 3. From within the separating column 4, isobutylene gas flows out of the top of the column and passes through a condenser 7 whereby it is cooled to liquid isobutylene finished product. Unreacted tertiary butyl alcohol is condensed by the column top condenser to form a condensate liquid, part of which is refluxed within the column and part of which flows out, returning to the reaction vessel. After passing through the distributor 3, the dehydrated extractant enters the separating column from the top of the column and, after extracting and separating the moisture from the rising gas, it flows to the bottom of the column. The bottom of the column is fitted with a heater 5. After the dehydrated extractant has absorbed water, it is sent to the dehydration column 6, where it is heated and the steam is removed from it. The dehydrated extractant is sent by the pump through the cooling unit 8, where it is cooled, and it is then returned to separating column 4. Operating continuously in this manner, tertiary butyl alcohol is cracked to become isobutylene and water, and isolate is yielded.

1. A method of preparing isobutylene using tertiary butyl alcohol, which includes the following steps sequentially: continuously adding the raw material: tertiary butyl alcohol to a reaction vessel containing a solid superacid catalyst sold on the market and heating until a rolling boil and cracking begins; the isobutylene gas produced by cracking and the boiling tertiary butyl alcohol gas flow together into a separating column in which the top contains a condenser and the bottom contains a heater; conducting the dehydrated extractant from the distributor into the separating column and performing separation, thereby causing the isobutylene gas to flow out of the top of the separating column and to be cooled by the condenser to yield liquid isobutylene finished product; under the action of the condenser at the top of the separating column, unreacted tertiary butyl alcohol is condensed to become a condensed liquid, part of which is refluxed within the column and part of which flows out and returns to the reaction vessel; from the distributor, the dehydrated extractant enters the separating column from the top and, after the water is separated by the extractant from the rising gas, it flows to the bottom of the column; after the dehydrated extractant has absorbed water, it is sent to a dehydration column where it is heated and steam is extracted from it, after which a pump is used to send it to the condenser to be cooled, and then it is returned to the separating column, where it continues to participate in the reaction.

2. Equipment for preparing isobutylene using tertiary butyl alcohol in accordance with the method of claim 1 comprising a reaction vessel (1) and a separating column (4), characterized in that the upper part of the said separating column (4) is fitted with a column top condenser (2) and the bottom of the column is fitted with a heater (5); the said reaction vessel (1) with a raw material feed inlet and the separating column (4) are connected using a pipe; one end of the dehydration column (6), which has an outlet, is connected to the heater (5) at the bottom of the separating column (4), and the other end, after passing through a cooling unit (8), is connected to a distributor (3) at the top of the separating column; a condenser (7) with an outlet is connected to the outside of the said column top condenser (2).
